(19) United States
(12) Patent Application Publication
(10) Pub. No.: US 2014/0371836 A1
(43) Pub. Date: Dec. 18, 2014

(54) EXPANDABLE SUPPORTIVE ENDOLUMINAL STENT GRAFT

(71) Applicants: Pierre Galvagni Silveira, Cacupe/Florianopolis/SC (BR); Andrea Piga Carboni, Jurere/Florianopolis/SC (BR)

(72) Inventors: Pierre Galvagni Silveira, Cacupe/Florianopolis/SC (BR); Andrea Piga Carboni, Jurere/Florianopolis/SC (BR)

(21) Appl. No.: 13/918,753

(22) Filed: Jun. 14, 2013

(51) Int. Cl. A61F 2/07; D05B 1/00

(52) U.S. Cl. CPC A61F 2/07; D05B 1/00
USPC 623/1.2; 112/475.06; 112/475.08

(57) ABSTRACT

An endoluminal prosthesis having a graft sleeve, a set of internal graft channels formed within the graft sleeve and a self-expanding wire stent. The graft sleeve forms a main fluid flow channel between a first open end and a second open end of the graft sleeve and includes an external surface and an internal surface. The self-expanding wire stent is coaxially mounted with the graft sleeve and affixed to the graft sleeve. The set of internal graft channels includes at least two internal graft channels parallel to the graft sleeve, each internal graft channel having an inner open end within the first open end of the graft sleeve and an outer open end within the second open end of the graft sleeve, thereby forming a set of fluid flow channels between the end openings of the graft sleeve.

### CROSS-REFERENCE TO RELATED APPLICATIONS

None

### STATEMENT REGARDING FEDERALLY SPONSORED RESEARCH OR DEVELOPMENT

None

### NAMES OF PARTIES TO A JOINT RESEARCH AGREEMENT

None

REFERENCE TO A “SEQUENCE LISTING”

None

### BACKGROUND OF THE INVENTION

This invention relates generally to tubular prostheses, such as grafts, stents, stent-graft and like for implantation within a human or animal body for the repair of damaged vessels, ducts or other physiological passageways, and to methods of making and using such apparatus.

The functional vessels of human and animal bodies, such as blood vessels and ducts, may occasionally weaken, increase in diameter and eventually rupture. For example, an abnormal dilatation of the wall of the aorta causes a sac, called aortic aneurysm. Upon further exposure to hemodynamic forces, such an aneurysm can rupture, with ensuing fatal hemorrhaging in a very short time.

One surgical intervention for weakened, aneurysmal or ruptured vessels involves the use of an endoluminal prosthesis, such as a stent graft, to provide some or all the functionality of the original, healthy vessel.

Briefly, a stent graft comprises two major components, a stent and a graft. The stent typically takes the form of a somewhat stiff tube-like structure inserted into an affected vessel and fixed in place. The stent may serve to maintain a patent vessel lumen, may serve as a structural support for the vessel, and/or may serve as attachment/seal for a graft. A graft typically takes the form of a flexible tube or sleeve which is at least somewhat fluid tight. When secured within a vessel using stents the graft becomes a surrogate vessel-within-a-vessel, and bears the brunt of the intravascular fluid pressure. It has become common practice to bridge damaged vessel segment using a sufficiently long graft secured within the vessel with stent segments.

This clinical approach, which is significantly less invasive than the traditional surgical procedure, is known as endovascular grafting. Stent grafts are used for treatment of vasculature in the human or animal body to bypass a repair or defect in the vasculature. For instance, a stent graft may be used to span an abdominal aortic aneurysm.

Complications arise, however, when vessel damage occurs near a vessel branch point, such as a mesenteric artery or a renal artery. Bypassing such an artery without providing blood flow into the branch artery can cause serious problems to the patient.

In order to overcome some or all of these drawbacks, branched endovascular prostheses have been proposed which provide a fenestration in the wall of a stent graft which, when the stent graft is deployed, is positioned over the opening to the branch vessel. Another stent graft can be deployed through the fenestration into the branch vessel to provide a blood flow path to the branch artery. Proper matching of the prosthesis to the proximal neck of the aortic vessel and the branching blood vessels is critical to the treatment of an aneurysm.

However a wide variation in vessel morphology has to be expected; for example, aortas vary in length, diameter and angulation between the renal artery region and the region of the aortic bifurcation. All possible combinations of these variables need to be considered, calling for large inventories of fenestrated prosthesis or for custom prosthesis.

Moreover, in certain conditions, endovascular grafting must be performed in a short period of time, not always compatible with the time needed for designing and manufacturing custom prosthesis.

Also a problem exists in mapping the vasculature so that a fenestration is positioned correctly in relation to the branch vessel when a custom stent graft is constructed. Where the position of a fenestration with respect to a branch vessel is offset when the stent graft is deployed, it may be difficult to deploy guide wires and catheters from the stent graft into the branch vessel to enable correct positioning of the branch vessel stent graft. Also when the fenestration is offset from the branch and a stent graft is deployed into the branch vessel from a main stent graft, the branch vessel stent graft may be kinked to such an extent that blood flow will not occur through it.

### DESCRIPTION OF THE BACKGROUND ART

More elaborate, multi-components devices are required to both shield the damaged vessel portion while maintaining blood flow through the main and branch vessels. Such devices are described in the following patents and references cited therein.

U.S. Pat. No. 5,632,772 describes a self-expanding endoluminal graft, preferably provided as a plurality of components that are deployed separately at the branching vessel location fitting in a telescoping manner.

U.S. Pat. No. 5,984,955 describes a system and method for endoluminal grafting of a main anatomical conduit and various branch conduits, comprising a primary graft with openings and branch grafts.

U.S. Pat. No. 5,653,743 describes a small bifurcated graft which may be placed in each hypogastric artery to maintain patency both to it and to the external iliac artery and the leg below.

U.S. Pat. No. 5,824,040 describes a branching endoluminal prosthesis for using in branching body lumen systems including trunk and branch lumens, comprising radially expandable trunk and branch portions, and radially expandable Y-connector portion.

U.S. Pat. No. 6,645,242 describes a bifurcated intravascular stent graft comprising a stent and a sleeve. The sleeve has an internal channel communicating with the side opening, thereby providing a branch flow channel from the main channel out through the side opening.

U.S. Appl. 2004/0193254 describes a prosthetic trunk lumen extending therethrough comprising a wall and an anastomosis in the wall, wherein a prosthetic trunk branch is disposed.

U.S. Appl. 2009/0048663 describes a system comprising a prosthetic device with a major lumen extending therethrough, a wall with one or more openings, and one or more branches extending into the major lumen.

The branching stent-graft structures of the prior art have generally comprised uniform structures, in which the branch fenestrations are substantially orthogonal to the aortic portion when the prosthesis is at rest. Although these straight branching prostheses are intended to deform somewhat to accommodate the branch angles of body lumen systems, the imposition of substantial axial bends due to an offset between branch and fenestration on known endovascular stent-grafts tends to cause folding, kinking, or wrinkling which occludes the lumens of the stent-grafts and degrades their therapeutic value. Still another disadvantage of known bifurcated stent-grafts is that even when they are flexed to accommodate varying branch geometries, the prosthetic bifurcation becomes distorted, creating an unbalanced flow to the branches. To overcome these limitations, it has often been necessary to limit these highly advantageous, minimally invasive endovascular therapies to patients having vascular geometries and abdominal aortic aneurysms which fall within very narrow guidelines.

Many of the prior-art devices are suitable for vessel branches where the branch vessel leaves the main vessel at a relatively small angle (less than about 45°, or example). For larger branching angles (as large as about 90° or even up to about 180°, for example) many prior art devices are not suitable. Such large branching angles occur at several potentially important repair sites (particularly along the abdominal aorta, at the renal arteries, celiac artery, superior and inferior mesenteric arteries, for example). Another drawback common to many devices of the prior-art is the need for transluminal access through the branch vessel from a point distal of the repair site. In many instances such access is either impossible (celiac artery, mesenteric arteries, renal arteries) or extremely difficult and/or dangerous (carotid arteries). Still other previous devices do not provide a substantially fluid-tight seal with the branch vessel, thereby partially defeating the purpose of the stent graft (i.e., shielding the repaired portion of the main vessel and/or branch vessel from intravascular fluid pressure).

It is therefore desirable to provide an endoluminal device to increase the number of vessel morphologies covered by a small number of prosthesis, especially in those cases where branched vessels are involved. It is also desirable to provide an endoluminal device which can be deployed within standard prosthesis in order to increase the vessel morphologies covered by these prosthesis.

### BRIEF SUMMARY OF THE INVENTION

The present invention provides radially expansible tubular prostheses, particularly grafts, stents, and stent-grafts, which are highly adaptable to varying luminal system geometries. The tubular prostheses, such as grafts, stents, stent-graft and like are for implantation within a human or animal body, secured in place by suture, stent or other suitable means, for the repair of damaged vessels, ducts or other physiological passageways. The prostheses of the present invention are suitable for a wide variety of therapeutic uses, including stenting of the vasculature, ureter, urethra, trachea, esophagus, biliary tract, and the like.

These prostheses will generally be radially expansible from a narrow diameter configuration to facilitate introduction into the body lumen, typically during surgical cutdown or percutaneous introduction procedures.

The prosthetic structures of the present invention will find their most immediate use as endovascular prostheses for the treatment of diseases of the vasculature, particularly aneurysms, stenoses, especially in those cases where the damaged or defected portion of the vasculature may include a branch vessel such as a mesenteric artery or a renal artery.

The prosthetic structures of the present invention will find their most immediate use as endoluminal prostheses being implanted in a vessel or lumen, or inside the vessel of an implanted prosthesis, in order to split or divide the vessel into multiple smaller vessels.

The prosthetic structures described herein below will find use in axially uniform cylindrical prostheses, in preassembled bifurcated prostheses, and as prosthetic modules which are suitable for selective assembly either prior to deployment, or in situ.

In one aspect of the invention there is an endoluminal prosthesis that comprises a graft sleeve, a set of internal graft channels formed within the graft sleeve and a self-expanding wire stent. The graft sleeve forms a main fluid flow channel between a first open end and a second open end of the graft sleeve and includes an external surface and an internal surface. The self-expanding wire stent is coaxially mounted with the graft sleeve and affixed to the graft sleeve. The set of internal graft channels includes at least two internal graft channels parallel to the graft sleeve, each internal graft channel having an inner open end within the first open end of the graft sleeve and an outer open end within the second open end of the graft sleeve, thereby forming a set of fluid flow channels between the end openings of the graft sleeve. The set of internal graft channels are obtained by repeatedly bending or folding the internal surface of the graft sleeve along a longitudinal line extending along the inner surface and connecting the internal surface along a segment extending along the longitudinal line by means of sewing or other mechanical means. The self-expanding wire stent and the first open end of the graft sleeve are adapted for engaging an endoluminal surface of a first segment of a main vessel and forming a substantially fluid-tight seal therewith; and the self-expanding wire stent and the second open end of the graft sleeve are adapted for engaging an endoluminal surface of a second segment of a main vessel and forming a substantially fluid-tight seal.

In an embodiment of the present invention, the endoluminal prosthesis comprises: a self-expanding wire stent and a graft sleeve. The graft sleeve comprises an upper tubular body defining a primary inlet, and a plurality of tubular legs, each leg defining an outlet, being the outlets of the legs in fluid communication with the inlet of the upper tubular body. The plurality of tubular legs are bended inside the upper tubular body, being the outlets of the plurality of tubular legs placed inside the primary inlet of the upper tubular body, defining a secondary inlet in the graft sleeve at the point where the plurality of tubular legs bended inside the upper tubular body, being the secondary inlet in fluid communication with the outlets of the plurality of legs. The self-expanding wire stent is coaxially arranged substantially internally to the upper tubular body and externally to the plurality of tubular legs, and it is affixed or operatively connected to the upper tubular body and to the plurality of tubular legs. The self-expanding wire stent and the primary inlet of the graft sleeve are adapted for engaging an endoluminal surface of a first segment of a main vessel and forming a substantially fluid-tight seal therewith; and the self-expanding wire stent and the secondary inlet of the graft sleeve are adapted for engaging an endoluminal surface of a second segment of a main vessel and forming a substantially fluid-tight seal.

### BRIEF DESCRIPTION OF THE SEVERAL VIEWS OF THE DRAWINGS

FIG. 1 is a schematic view of the multi-port endoluminal prosthesis in accordance with the present invention

FIG. 2 is a schematic view of a prior art stent

FIG. 3 is a perspective view of the multi-port endoluminal prosthesis in accordance with the present invention

FIG. 4 is a schematic view of a tubular sleeve

FIG. 5 shows the tubular sleeve being split or divided along a longitudinal line

FIG. 6 shows the tubular sleeve split or divided along a longitudinal line forming two tubular bodies

FIG. 7 shows the tubular sleeve coaxially mounted internally to the stent

FIG. 8 shows a schematic view of the tubular sleeve with the stent positioned over the tubular sleeve.

FIG. 9 shows an alternative embodiment of the multi-port endoluminal prosthesis

FIG. 10 shows an alternative embodiment of the multi-port endoluminal prosthesis

FIG. 11 shows an alternative embodiment of the multi-port endoluminal prosthesis

FIG. 12 shows an alternative embodiment of the multi-port endoluminal prosthesis

FIG. 13 shows an alternative embodiment of the multi-port endoluminal prosthesis with multiple tubular bodies

FIG. 14 is a schematic view of a guide wire advanced through the iliac, the aorta and the stent graft

FIG. 15 is a schematic view of a guide catheter advanced through the iliac, the aorta and the stent graft

FIG. 16 is a schematic view of a rigid guide wire advanced through the iliac, the aorta and the stent graft

FIG. 17 is a schematic view of a delivery catheter advanced through the iliac, the aorta and the stent graft

FIG. 18 shows the multi-port endoluminal prosthesis deployed inside a stent graft

FIG. 19 is a schematic view of a guide wire advanced through the subclavian, the aorta, stent graft, the multi-port endoluminal prostheses and the renal artery

FIG. 20 is a schematic view of a guide catheter advanced through the subclavian, the aorta, stent graft, the multi-port endoluminal prostheses and the renal artery

FIG. 21 is a schematic view of a stiffer guide wire advanced through the subclavian, the aorta, stent graft, the multi-port endoluminal prostheses and the renal artery

FIG. 22 is a schematic view of a delivery catheter advanced through the subclavian, the aorta, stent graft, the multi-port endoluminal prostheses and the renal artery

FIG. 23 shows a stent graft deployed between renal artery and the multi-port endoluminal prosthesis.

FIG. 24 shows stent grafts deployed between renal artery, superior mesenteric artery, renal artery and the multi-port endoluminal prosthesis.

FIG. 25 shows the multi-port endoluminal prosthesis deployed inside a stent graft

FIG. 26 shows stent grafts deployed between renal artery, superior mesenteric artery, renal arteries and the multi-port endoluminal prostheses.

FIG. 27 shows the multi-port endoluminal prostheses deployed inside a stent graft.

FIG. 28 shows the multi-port endoluminal prostheses deployed inside a iliac artery.

### DETAILED DESCRIPTION OF THE INVENTION

The present invention relates to endoluminal vascular prostheses and methods of deploying such prostheses. Stylized drawings of human body parts are shown in frontal view, where the left side of the drawing corresponds to the right side of human body.

It will be understood that the embodiments of the present invention described herein are illustrative of some of the applications of the principles of the present invention. Various modifications may be made by those skilled in the art without departing from the true spirit and scope of the invention.

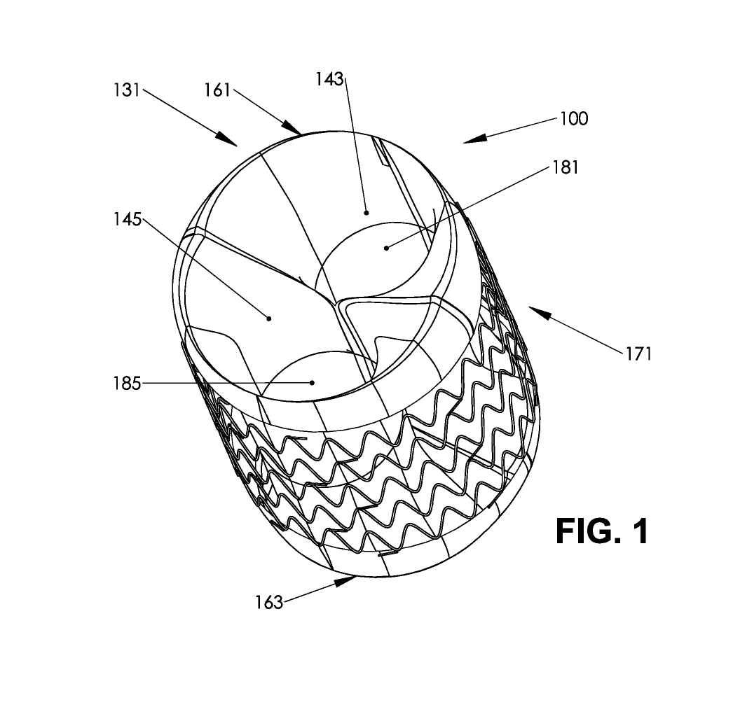

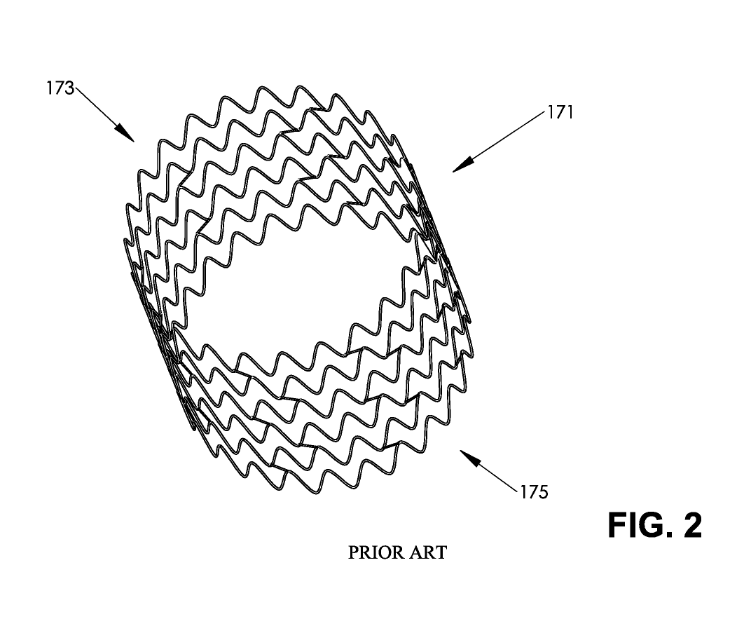

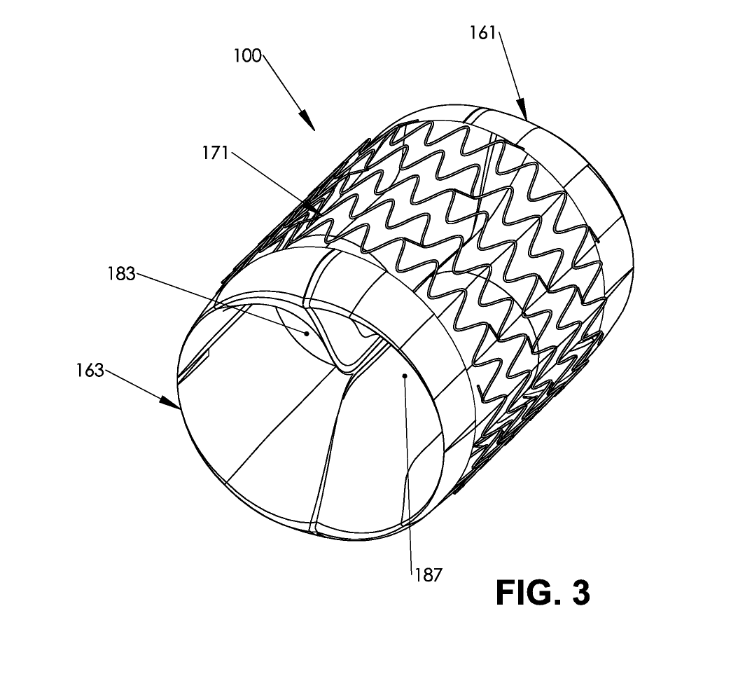

A multi-port endoluminal prosthesis, generally referred to as numeral 100, according to the present invention is illustrated in FIGS. 1 and 3. The multi-port endoluminal prosthesis 100 comprises a graft 131 and a stent 171. The stent 171 comprises a first end 173 and a second end 175, as shown in FIG. 2.

The graft 131 comprises a hollow inlet 161 and a hollow outlet 163 as shown in FIG. 3. The graft 131 comprises a first tubular body 143 and a second tubular body 145 as shown in FIG. 1 and FIG. 3 The first tubular body 143 includes a first hollow inlet 181 and a first hollow outlet 183. The second tubular body 145 includes a second hollow inlet 185 and a second hollow outlet 187. The first and second tubular bodies 143 and 145 are in fluid communication with the hollow inlet 161 and the hollow outlet 163.

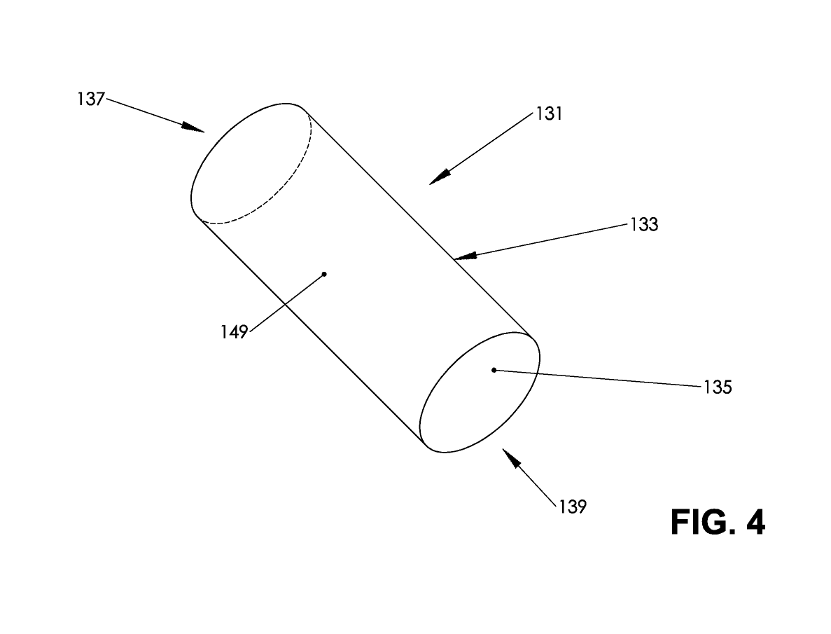

The multi-port endoluminal prosthesis 100 may be obtained with the method described below and illustrated in FIG. 4-8. It will be appreciated by those skilled in the art that other modifications could be made or different methods could be used to obtain the prosthesis of the invention without deviating from its spirit and scope as claimed.

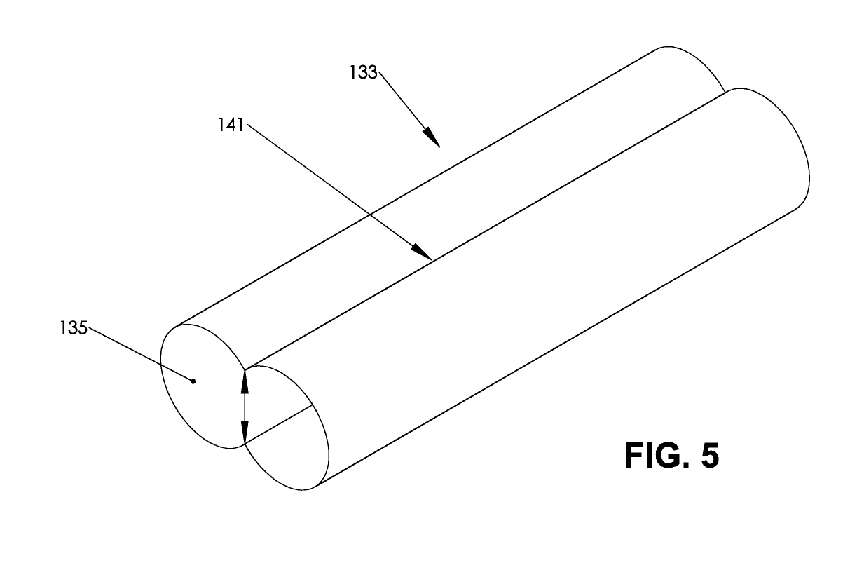

The graft 131 of the multi-port endoluminal prosthesis 100 may be obtained from a tubular sleeve 133, as shown in FIG. 4-8. The sleeve 133 is preferably made of woven polyester graft material, like Dacron, polytetrafluoroethylene (PTF), expanded PTFE, and other synthetic materials known to those of skill in the art. The sleeve 133 comprises an inner surface 135, an external surface 149, a first open end 137 and a second open end 139, as illustrated in FIG. 4. The inner surface 135 of the sleeve 133 can be bent along a longitudinal line 141 extending along the inner surface 135 as shown by the arrows in FIG. 5.

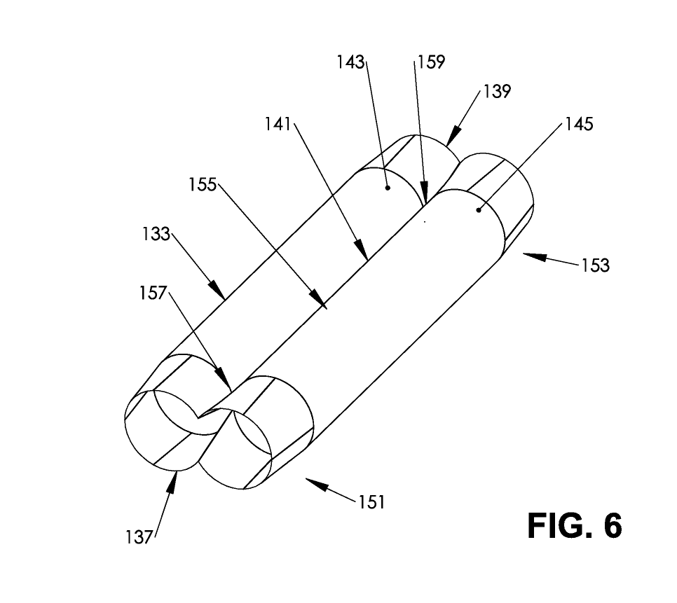

A portion of the longitudinal line 141, herein referred to as a connection segment 155, comprises a first end 157 and a second end 159 as showed in FIG. 6. The inner surface 135 can be connected along the connection segment 155, as shown in FIG. 6, by means of sewing or any other mechanical means that provide sealing between the first and second tubular bodies 143 and 145. The connection segment 155 comprises a first end 157 and a second end 159.

The sleeve 133 is split or divided into the first tubular body 143 and the second tubular body 145, not necessarily of the same diameter, in the central portion, while the first open end 137 and the second open end 139 keep a one-vessel configuration, as shown in FIG. 6.

A first unsplit or undivided portion 151 of the sleeve 133 is therefore delimited from the first open end 137 and the first end 157 of the connection segment 155 of the inner surface 135. A second unsplit or undivided portion 153 is therefore delimited from the second open end 139 and the second end 159 of the connection segment 155 of the inner surface 135.

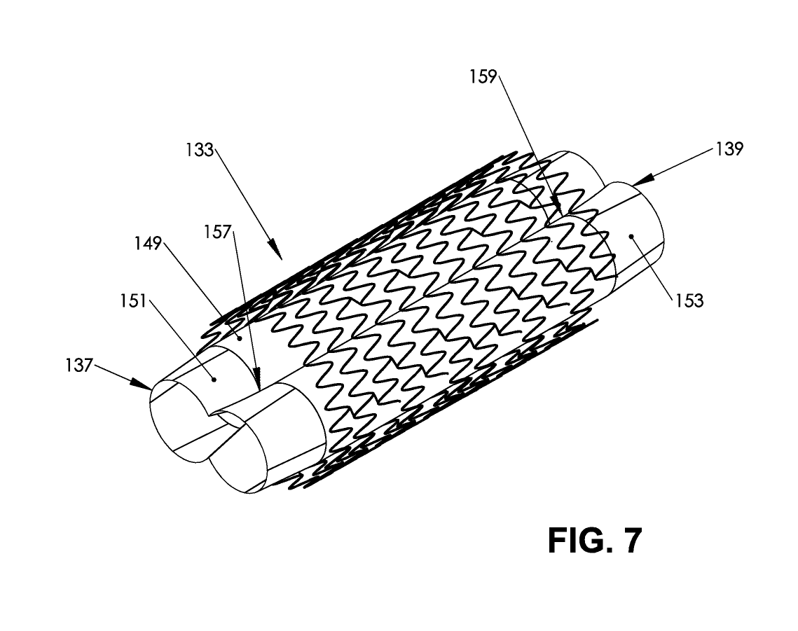

The stent 171 is preferably a self-expanding stent, ideally comprising a shape memory alloy such as super-elastic Nitinol, or the like. The tubular sleeve 133 is coaxially mounted internally to the stent 171, as shown in FIG. 7. The sleeve 133 may be operatively connected to the stent 171 by means of suturing the external surface 149 to the stent 171, or by any other mechanical means.

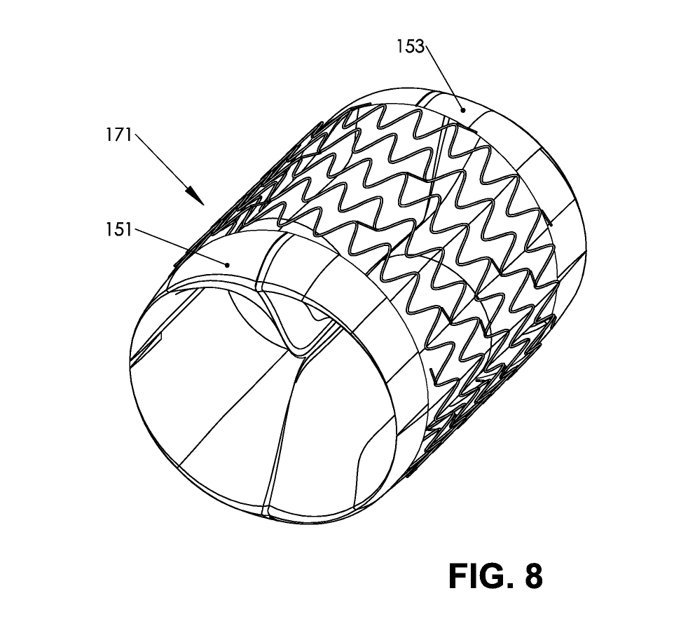

The first unsplit or undivided portion 151 is bent over the stent 171, recovering it for some extension, as shown in FIG. 8. The first unsplit or undivided portion 151 may be operatively connected to the stent 171 by means of suturing. The second unsplit or undivided portion 153 is bent over the stent 171, recovering it for some extension, as shown in FIG. 8. The second unsplit or undivided portion 153 may be operatively connected to the stent 171 by means of suturing.

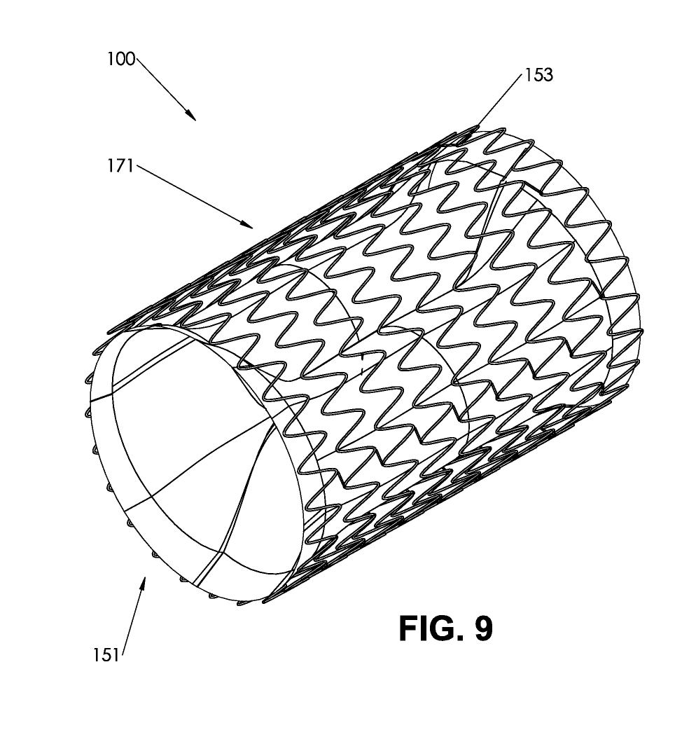

In an alternative embodiment of the present invention, the first unsplit or undivided portion 151 and second unsplit or undivided portion 153 may be directly connected to the stent 171, without bending over the stent 171, by means of suturing, adhesive or encapsulating the stent 171 as shown in FIG. 9.

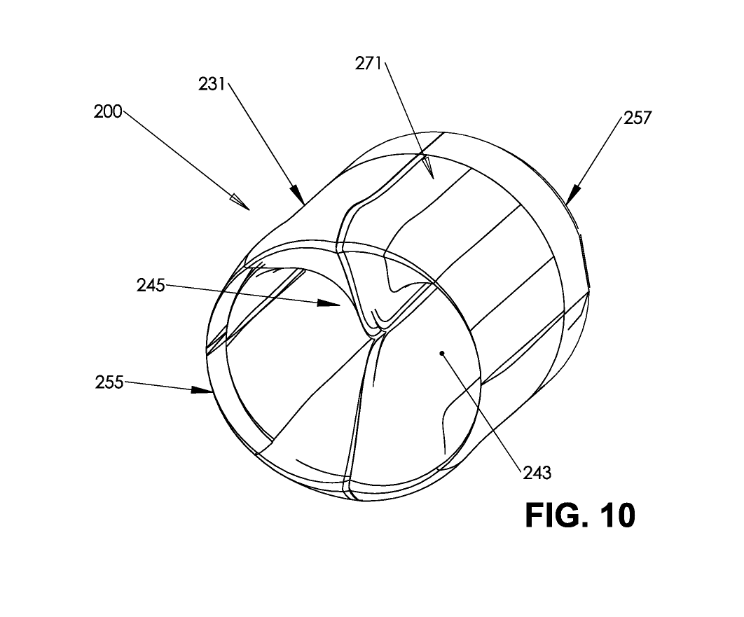

Referring to FIG. 10-12 an alternative embodiment of the multi-port endoluminal prosthesis is described, generally to as numeral 200. The multi-port endoluminal prosthesis 200 comprises a graft 231 and a stent 271.

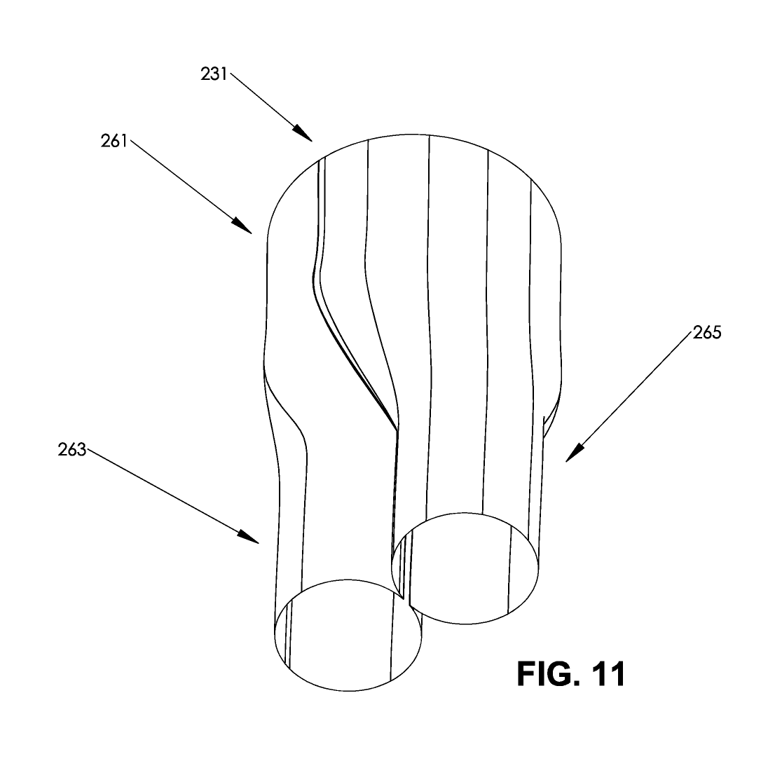

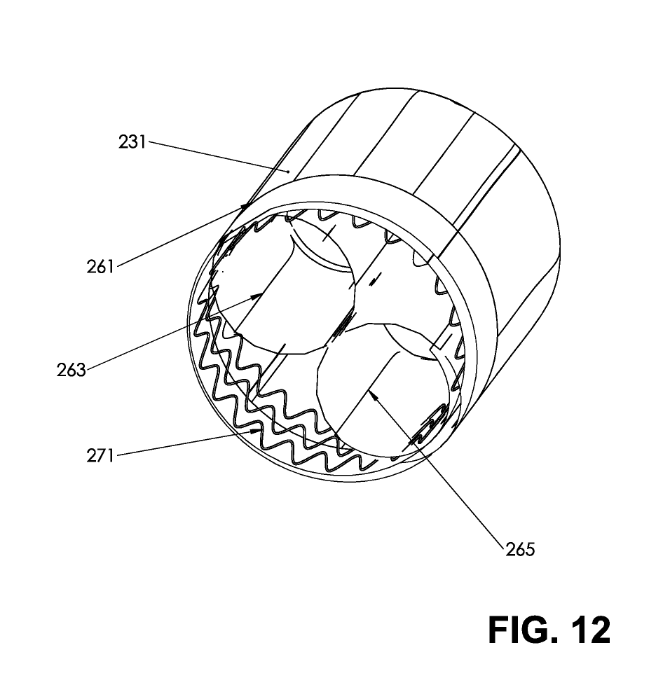

The graft 231 comprises a hollow inlet 255 and hollow outlet 257 as shown in FIG. 10. The graft 231 comprises a trunk component 261, a first leg 263 and a second leg 265, as shown in FIG. 11. The first leg 263 and the second leg 265 are in fluid communication with the hollow inlet 255. The first leg 263 and the second leg 265 are bended inside the trunk component 261 as shown in FIG. 12.

The stent 271 is preferably a self-expanding stent, ideally comprising a shape memory alloy such as super-elastic Nitinol, or the like. The graft 231 is coaxially mounted internally the stent 271, as shown in FIG. 12. The graft 231 may be operatively connected to the stent 271 by means of suturing or any other mechanical means.

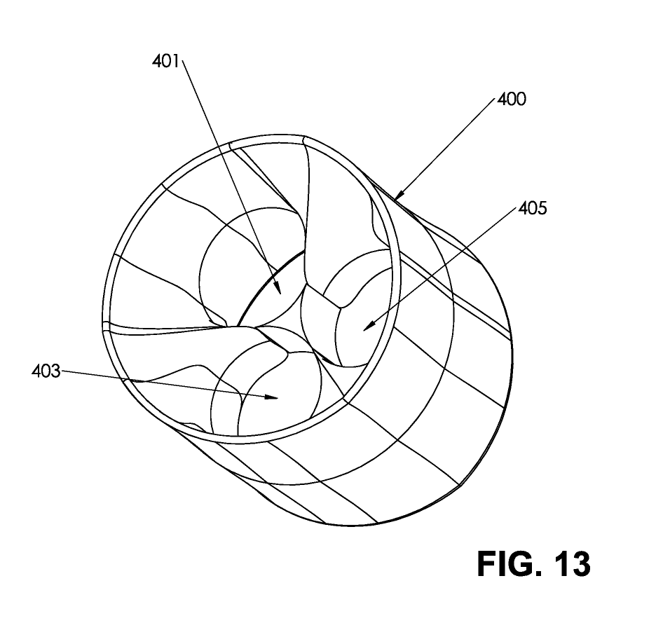

In an alternative embodiment the number of tubular bodies may vary to three or more. FIG. 13 shows a three legs multi-port endoluminal 400 prosthesis comprising a first tubular body 401, a second tubular body 403 and a third tubular body 405.

The multi-port endoluminal prostheses of the present invention is particularly well-suited for repair of main vessel segments where one or more branch vessels leave the main vessel at an angle approaching 90°. Previous bifurcated stent graft devices enable repairs where a branch vessel leaves the main vessel at a substantially smaller angle of less than about 45°. This limitation in the prior art does not allow for repair at several potentially important locations within the vasculature, ureter, urethra, trachea, esophagus, biliary tract, and the like.

Other previous devices enable repair at such high angled branches only when transluminal access to a distal portion of the branch vessel is possible. In many instances such access is either impossible (celiac artery, mesenteric arteries, renal arteries) or extremely difficult and/or dangerous (carotid arteries). Still other previous devices do not provide a substantially fluid-tight seal with the branch vessel, thereby partially defeating the purpose of the stent graft (i.e., shielding the repaired portion of the main vessel and/or branch vessel from intravascular fluid pressure).

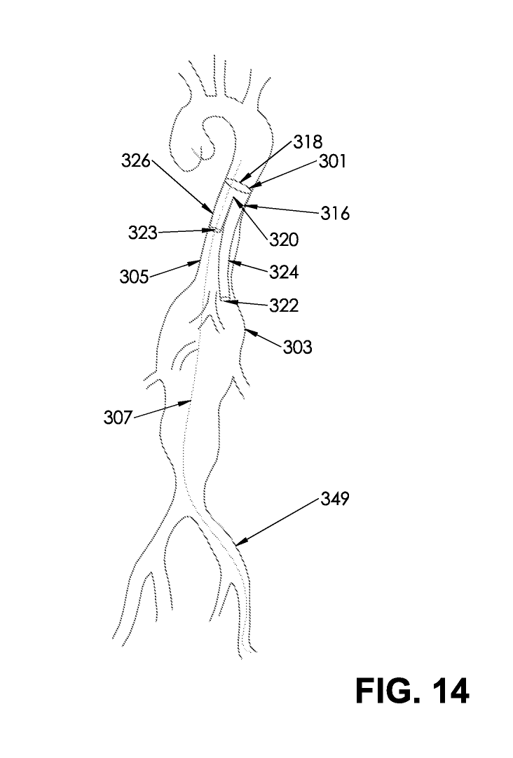

The prostheses of the present invention, in contrast, addresses these issues. As shown in FIG. 14, a bifurcated stent graft 301 (for instance as the stent graft described in patent U.S. 2011/0130828) may be delivered transluminally to repair an aneurysmatic site 303 of a main vessel 305 (for instance by the method as described in U.S. 2011/0130828). The bifurcated stent graft 301 in FIG. 14 has an upper tubular body 316 which defines a hollow inlet 318, and a lower bifurcation 320 which includes a first tubular leg 324 defining a first outlet 322 and a second tubular leg 326 which is shorter than the first tubular leg 324 and defines a second outlet 323. The first and second tubular legs 324 and 326 are in fluid communication with the hollow inlet 318.

As shown in FIG. 14 a flexible guide wire 307 extends through the second tubular leg 326 and second outlet 323 of the lower bifurcation 320 of the bifurcated stent graft 301, extending to an iliac artery 349 so that it can function to guide the advancement of a guide catheter through the iliac artery 349 and into the second tubular leg 326 of the bifurcated stent graft 301 in order to guide a stiffer guide wire which is distally advanced into the second tubular leg 326 of the bifurcated stent graft 301. As further discussed below, the guide catheter is then removed and a catheter delivery system is advanced over the stiffer guide wire through the iliac artery 349 and into the second tubular leg 326 of the stent graft within the aorta 350 to facilitate delivery and deployment of the endoluminal multi-port prostheses therein.

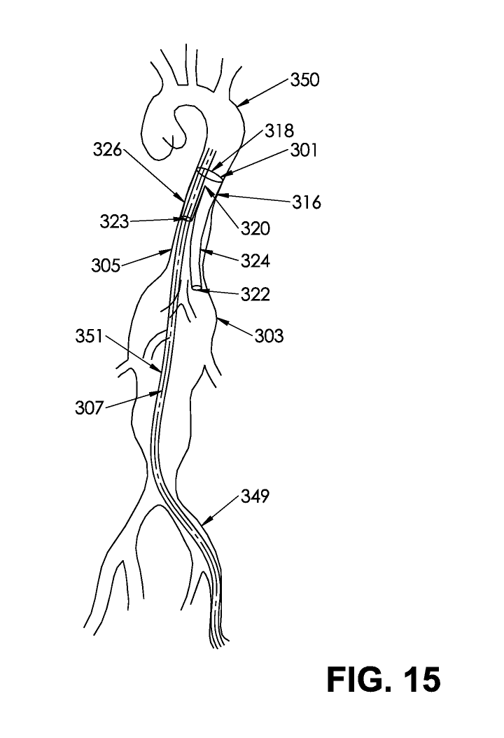

Turning to FIG. 15, a guide catheter 351 is advanced over the flexible guide wire 307. The flexible guide wire 307 guides the guide catheter 351 through the iliac artery 349, through a portion of the aorta 350, and into the second tubular leg 326 of the bifurcated stent graft 301. The surgeon can advance the guide catheter 351 over the guide wire 307, and the guide wire 307 will guide the distal end of the guide catheter 351 into the second outlet 323 of the second tubular leg 326.

Once the guide catheter 351 is disposed inside the second tubular leg 326 of the bifurcated stent graft 301, the flexible guide wire 307 is retracted proximally through the second tubular leg 326, the aorta 350, the iliac artery 349, and out of the patient, while the guide catheter 351 remains advanced within the second tubular leg 326.

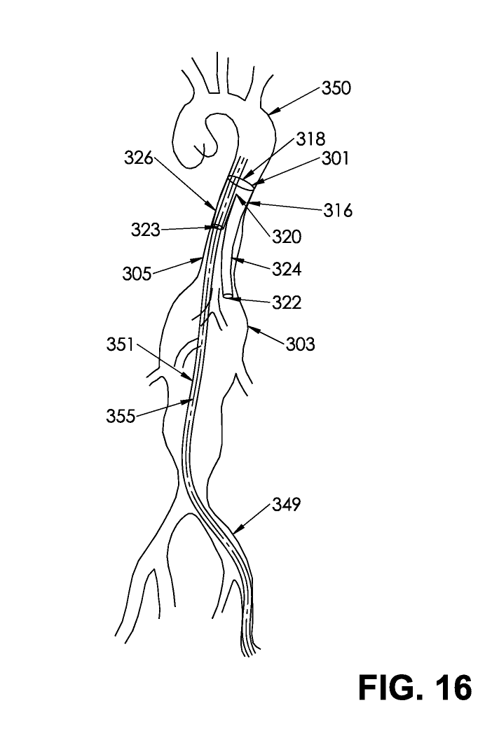

A stiffer guide wire 355 (FIG. 16) may then be advanced through the guide catheter 351 to the distal end of the guide catheter 351 inside the second tubular leg 326, and the guide catheter 351 may be removed, leaving the stiffer guide wire 355 in place.

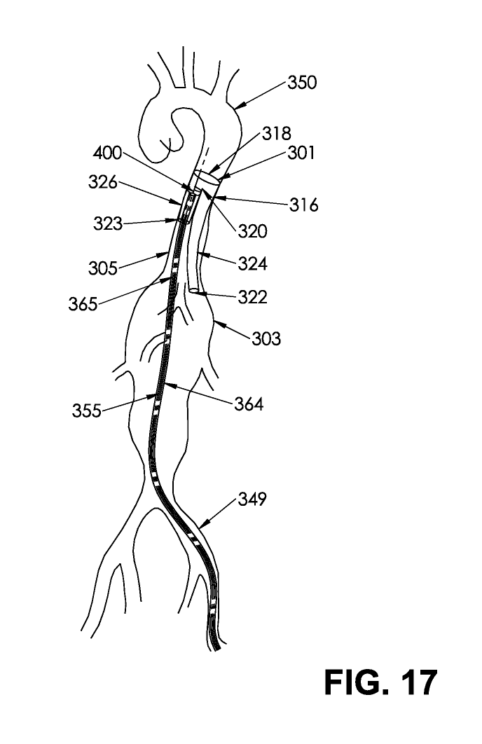

Turning to FIG. 17, with the stiffer guide wire 355 in place, a catheter delivery system 364 may be provided with the multi-port endoluminal prostheses 400 with three internal channels and a stent delivery device 365 and may be introduced into the patient and distally advanced over the stiffer guide wire 355, which guides the catheter delivery system 364 through the iliac artery 349 and aorta 350, and into the second tubular leg 326 of the bifurcated stent graft 301.

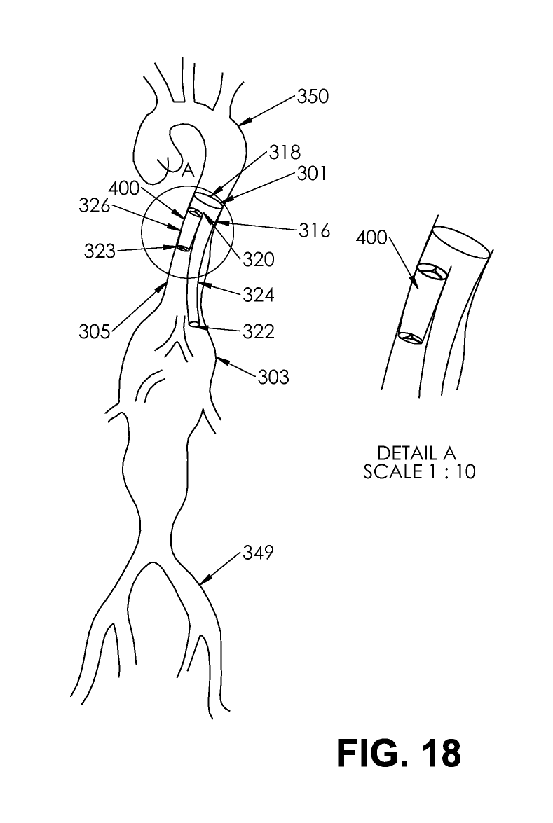

The multi-port endoluminal prostheses 400 can then be deployed from the catheter delivery system 364 inside the second tubular leg 326 of the bifurcated stent graft 301 (e.g., the delivery catheter is refracted proximally relative to the stent delivery device 365, which is held longitudinally fixed, which deploys the multi-port endoluminal prostheses 400). The stiffer guide wire 355 may be removed from the patient before or after deployment of the multi-port endoluminal prostheses 400. The catheter delivery system 364 can be operated to deploy the multi-port endoluminal prostheses 400 between the second outlet 323 of the second tubular leg 326 and the lower bifurcation 320 as shown in FIG. 18.

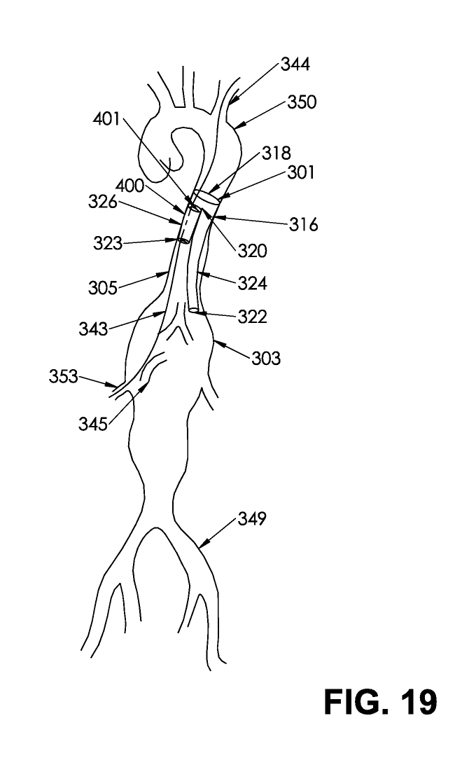

Turning to FIG. 19 a thin guide wire 343 is inserted through a left subclavian artery 344 and advanced through a portion of the aorta 350, through the hollow inlet 318 of the bifurcated stent graft 301, through the second tubular leg 326, through the first tubular body 401 of the multi-port endoluminal prostheses 400 and through the right renal artery 353.

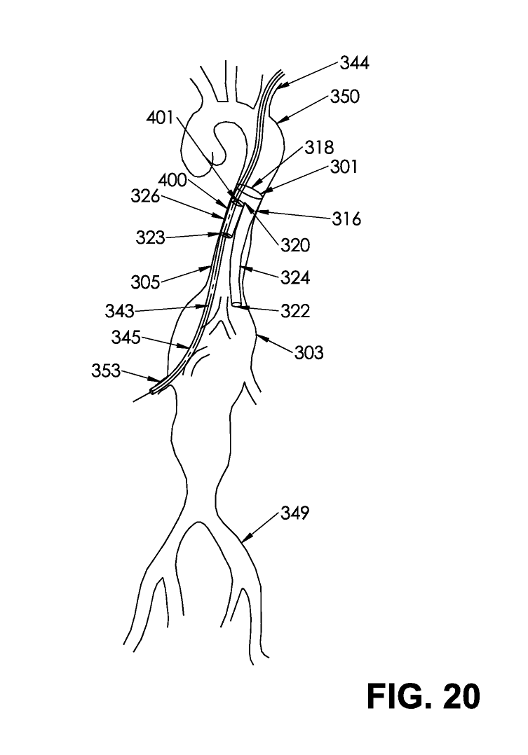

Turning to FIG. 20, a guide catheter 345 is advanced over the thin guide wire 343. The thin guide wire 343 guides the guide catheter 345 through the left subclavian artery 344, through a portion of the aorta 350, through the hollow inlet 318 of the bifurcated stent graft 301, through the second tubular leg 326, through the first tubular body 401 of the multi-port endoluminal prostheses 400 and through the right renal artery 353. The surgeon can advance the guide catheter 345 over the thin guide wire 343, and the thin guide wire 343 will guide the distal end of the guide catheter 345 into the right renal artery 353 as shown in FIG. 20.

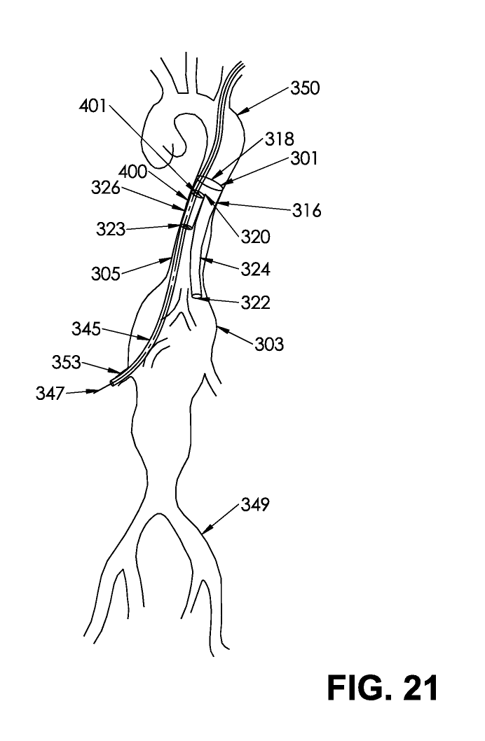

Once the guide catheter 345 is disposed inside the right renal artery 353 the thin guide wire 343 is retracted, while the guide catheter 345 remains advanced within the right renal artery 353. A stiffer guide wire 347 (FIG. 21) may then be advanced through the guide catheter 345 to the distal end of the guide catheter 345 inside the right renal artery 353 and the guide catheter 345 may be removed, leaving the stiffer guide wire 347 in place.

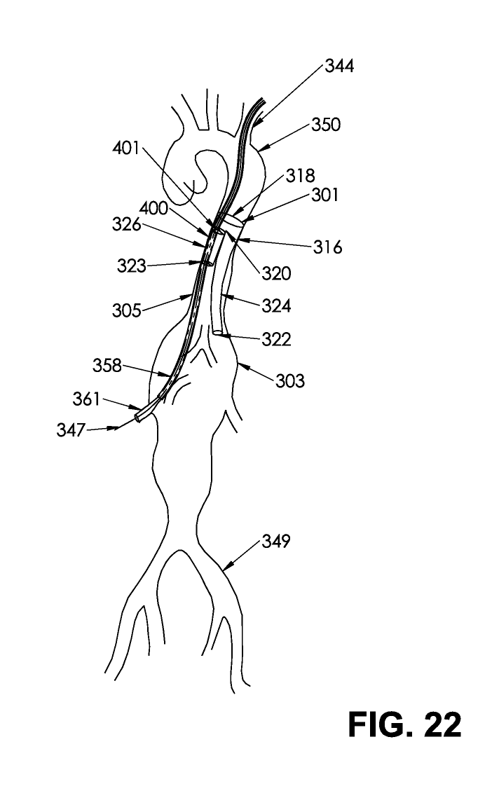

Turning to FIG. 22, a catheter delivery system 358 provided with a stent graft 361 (like a Viabhan) is advanced over the stiffer guide wire 347 through the left subclavian artery 344, through a portion of the aorta 350, through the hollow inlet 318 of the bifurcated stent graft 301, through the second tubular leg 326, through the first tubular body 401 of the multi-port endoluminal prostheses 400 and through the right renal artery 353.

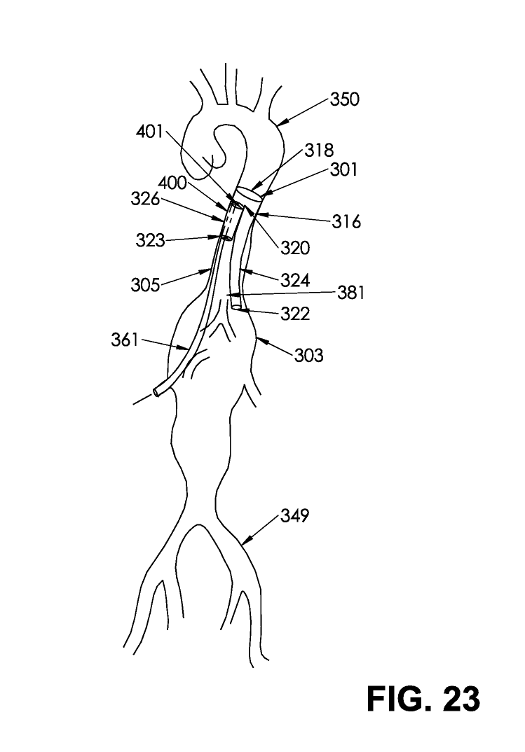

The stent graft 361 can then be deployed from the catheter delivery system 358 inside the right renal artery 353. (e.g., the delivery catheter is refracted proximally relative to the stent delivery device 363, which is held longitudinally fixed, which deploys the first stent graft 361). The stiffer guide wire 347 may be removed from the patient before or after deployment of the first stent graft 361. The catheter delivery system 358 can be operated to deploy the stent graft 361 between the right renal artery 353 and the first tubular body 401 of the multi-port endoluminal prostheses 400, as shown in FIG. 23.

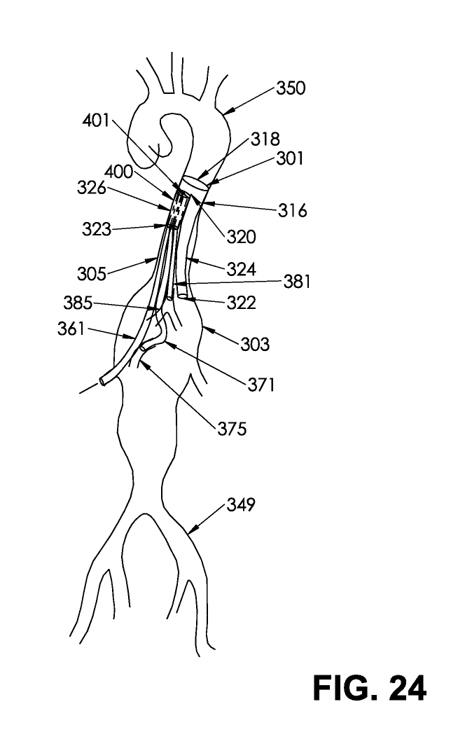

Using the same method described above, by means of guide wires, guide catheters and stent delivery devices, a stent graft 371 can be deployed between the superior mesenteric artery 375 and the second tubular body 403 of the multi-port endoluminal prostheses 400, as shown in FIG. 24. Similarly a stent graft 381 may be deployed between the celiac trunk 385 and the third tubular body 405 of the multi-port endoluminal prostheses 400.

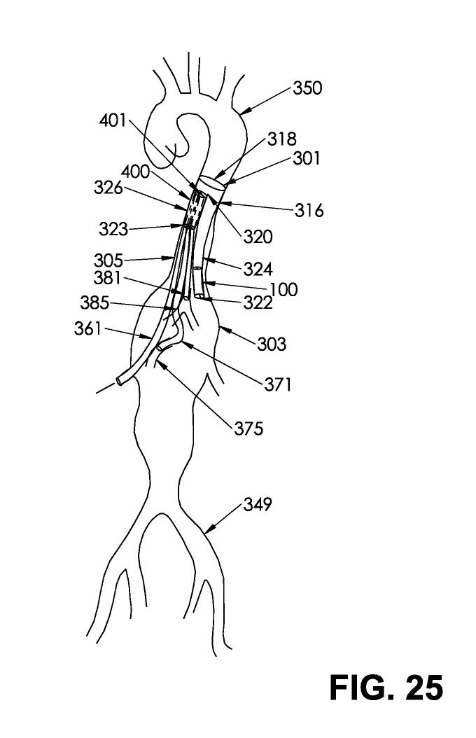

With a similar procedure, by means of guide wires, guide catheters and stent delivery devices the multi-port endoluminal prostheses 100 comprising the first tubular body 143 and the second tubular body 145 may be deployed inside the first tubular leg 324 of the bifurcated stent graft 301 between the first outlet 322 of the first tubular leg 324 and the lower bifurcation 320 as shown in FIG. 25.

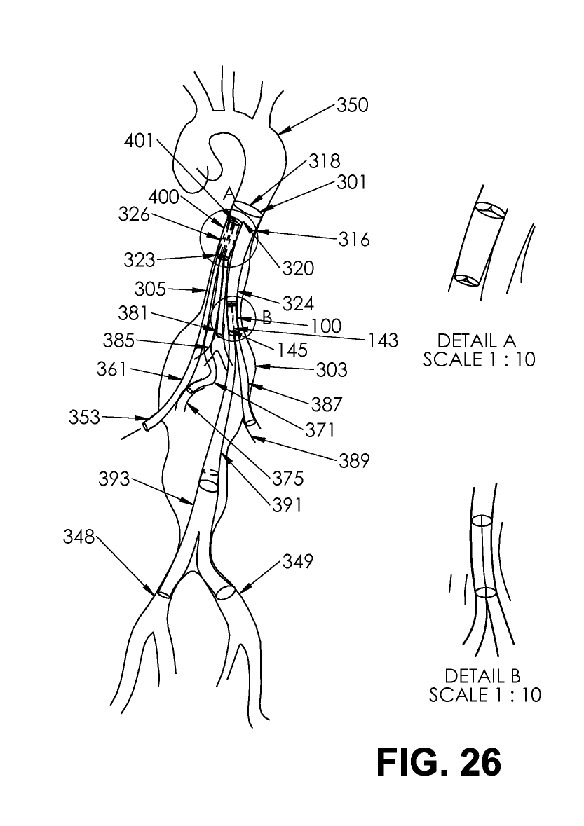

As shown in FIG. 26 a stent graft 387 can be deployed between left renal artery 389 and the first tubular body 143 of the multi-port endoluminal prostheses 100, by means of guide wires, guide catheters and stent delivery devices. A stent graft 391 can be deployed in the second tubular body 145 of the multi-port endoluminal prostheses 100 and a bifurcated stent graft 393 can successively be deployed between the stent graft 391 and the iliac arteries 348 and 349, as shown in FIG. 25

It will be appreciated that the stent grafts 361, 371, 381, 387, 391 and 393 together with the multi-port prosthesis 400 and 100 will now define passageways for blood flow from the aorta 350 upstream of the aneurysmatic site 303 to the left renal arteries 389 and the right renal arteries 353, to the superior mesentheric artery 375, to the celiac trunk 385 and to the iliac arteries 348 and 349, downstream of the aneurysmatic site 303 while excluding the damaged or otherwise unhealthy portion (e.g., the aneurysmatic site 303) of the aorta 350.

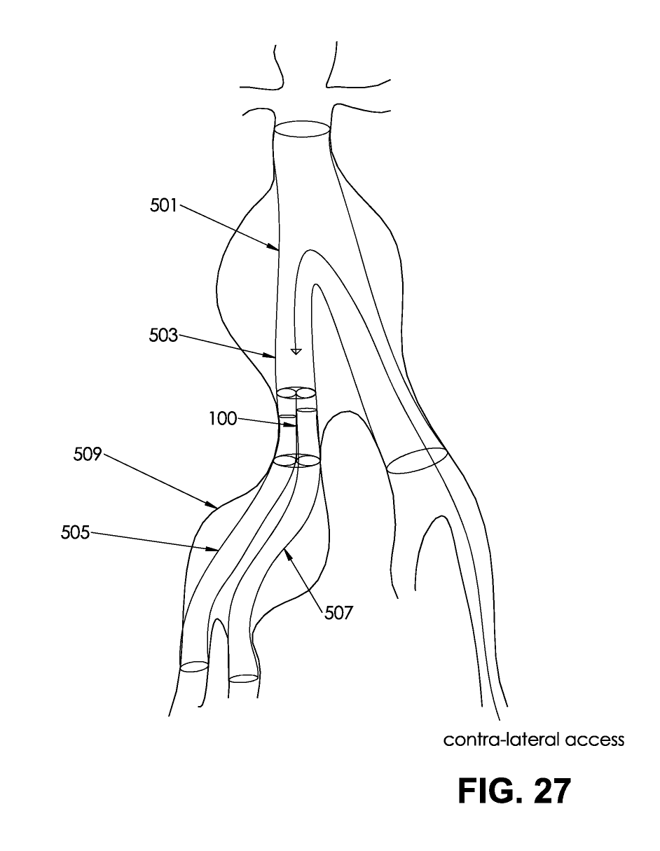

In FIG. 27 a stent graft 501 is deployed close to the iliac bifurcation 510. The multi-port endoluminal prosthesis 100 is deployed in a leg 503 of a bifurcated stent graft 501. In case of aneurysm of distal part of external iliac artery 509 contralateral access is gained and two stent grafts 505 and 507 are deployed between the multi-port endoluminal prosthesis 100 and the external and internal iliac arteries, as shown in FIG. 27.

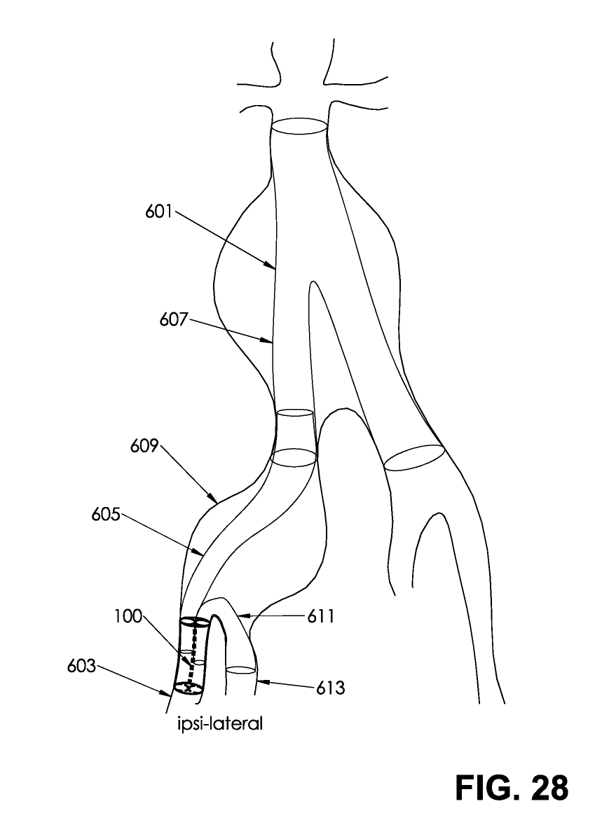

In FIG. 28 a stent graft 601 is deployed close to the iliac bifurcation 510. The multi-port endoluminal prosthesis 100 is deployed in the external iliac artery 603. In case of aneurysm 609 of distal part of external iliac artery 603 ipsilateral access is gained and a stent graft 605 is deployed between a leg 607 of the stent graft 601 and the external iliac artery 603. A second stent graft 611 is deployed between the multi-port endoluminal prosthesis 100 and the internal iliac artery, as shown in FIG. 27.

Additional stent grafts may be applied to one or more blood vessels as needed. There have been described and illustrated herein several embodiments of an apparatus and a method of repairing abdominal aortic aneurysms. While particular embodiments of the invention have been described, it is not intended that the invention be limited thereto, as it is intended that the invention be as broad in scope as the art will allow and that the specification be read likewise. It is also intended that all the embodiments illustrated herein may be used in the applications presented. Thus, while particular shaped and sized stent grafts have been disclosed, it will be appreciated that other shapes and sizes may be used as well. It will therefore be appreciated by those skilled in the art that yet other modifications could be made to the provided invention without deviating from its spirit and scope as claimed.

What is claimed is:

1. An expandable supportive endoluminal stent graft comprising:
a graft sleeve forming a main fluid flow channel between a first open end and a second open end of said graft sleeve and including an external surface and an internal surface;
a self-expanding wire stent coaxially mounted on said graft sleeve and affixed to said graft sleeve, wherein the self-expanding wire stent and the first open end of the graft sleeve are adapted for engaging an endoluminal surface of a first segment of a main vessel and forming a substantially fluid-tight seal therewith and the self-expanding wire stent and the second open end of the graft sleeve are adapted for engaging an endoluminal surface of a second segment of a main vessel and forming a substantially fluid-tight seal; and
a set of internal graft channels formed within the graft sleeve and separated by a partition, wherein the set of internal graft channels comprises at least two internal graft channel parallel to the graft sleeve, each internal graft channel having inner and outer ports within the ends of graft sleeve, thereby forming a set of fluid flow channels between the end openings of the graft sleeve.

2. The expandable supportive endoluminal stent graft according to claim 1, wherein the set of internal graft channels are obtained repeatedly folding the internal surface of the graft sleeve along a longitudinal line extending along the inner surface and connecting the internal surface along a segment extending along the longitudinal line by means of sewing or other mechanical means.

3. An expandable supportive endoluminal stent graft comprising:
a self-expanding wire stent, wherein the self-expanding wire stent is coaxially arranged substantially internally to an upper tubular body and externally to a plurality of tubular legs, said self-expanding wire stent being affixed to the upper tubular body and to the plurality of tubular legs and wherein the self-expanding wire stent and the primary inlet of the graft sleeve are adapted for engaging an endoluminal surface of a first segment of a main vessel and forming a substantially fluid-tight seal therewith and wherein the self-expanding wire stent and the secondary inlet of the graft sleeve are adapted for engaging an endoluminal surface of a second segment of a main vessel and forming a substantially fluid-tight seal;
a graft sleeve comprising an upper tubular body defining a primary inlet, and a plurality of tubular legs, each leg defining an outlet in fluid communication with the inlet of the upper tubular body and wherein the plurality of tubular legs are bent inside the upper tubular body, being the outlets of the plurality of tubular legs placed inside the primary inlet of the upper tubular body, defining a secondary inlet in the graft sleeve at the point where the plurality of tubular legs bended inside the upper tubular body, being the secondary inlet in fluid communication with the outlets of the plurality of legs.

4. A method of producing an expandable supportive endoluminal stent graft comprising:
folding a tubular sleeve along a longitudinal line extending along the inner surface of the sleeve and wherein the tubular sleeve is repeatedly folded and operatively connected along a set of longitudinal lines, forming a plurality of inner flow channels and wherein a portion of the tubular sleeve is bent over the stent, to cover a portion of the ends and wherein the tubular sleeve is operatively connected to the stent at the terminal ends of the tubular sleeve by means of suturing or encapsulating the stent;
operatively connecting the inner surface along a portion of the longitudinal line by means of sewing or other mechanical means, forming a set of inner flow channels;
mounting the tubular sleeve coaxially to a stent; and
operatively connecting the tubular sleeve to the stent by means of suturing or by other mechanical means.

5. A method of producing an expandable supportive endoluminal stent graft according to claim 4, comprising:
(a) a stent graft comprising an hollow inlet and an hollow outlet, a trunk component, a first leg and a second leg in fluid communication the hollow inlet;
(b) folding the first leg and the second leg inside the trunk component; and
(c) mounting a stent coaxially inside the trunk component and outside the first and the second leg.
